(19) United States
(12) Patent Application Publication
(10) Pub. No.: US 2022/0125668 A1
(43) Pub. Date: Apr. 28, 2022

(54) MULTI-PRONGED INNER STABILIZER CONFIGURATION FOR DERMA ROLLER APPARATUS

(71) Applicant: Hillary Hayman, Los Angeles, CA (US)

(72) Inventor: Hillary Hayman, Los Angeles, CA (US)

(21) Appl. No.: 17/079,144

(22) Filed: Oct. 23, 2020

(51) Int. Cl. A61H 15/00

(52) U.S. Cl. CPC A61H 15/0092; A61H 2015/0014

(57) ABSTRACT

A derma roller apparatus includes a handle, a plurality of inner stabilizers, and a head connected to the handle via the plurality of inner stabilizers. The head includes an axial member positioned transverse to the handle. Furthermore, the head includes a left geometrically-shaped member positioned around the axial member to the left of the plurality of inner stabilizers. Additionally, the head includes a first plurality of needles situated around the left geometrically-shaped member. Moreover, the head includes a right geometrically-shaped member positioned around the axial member to the right of the plurality of inner stabilizers. Also, the head includes a second plurality of needles situated around the right geometrically-shaped member.

### BACKGROUND

### 1. Field

This disclosure generally relates to skincare apparatuses for the skin around the eyes and face of a user. More particularly, the disclosure relates to a multi-pronged inner stabilizer configuration for a derma roller apparatus.

### 2. General Background

The skin around the eyes and face are often viewed as an early indicator of aging. Accordingly, the quest for youthful skin around the eyes has spawned many skincare products and treatments over the course of many centuries; these products and treatments range from non-invasive (e.g., topical eye creams) all the way to extremely invasive (e.g., plastic surgery). Yet, the efficacy of a particular product and treatment is not necessarily correlated to an increased level of invasiveness. In fact, invasive procedures, such as plastic surgery or dermatological procedures, sometimes lead to significant disfigurations.

Various apparatuses have been developed for at-home use to promote skincare rejuvenation around the eyes and on the face, but many of these apparatuses also have questionable efficacy. Furthermore, some of these apparatuses are difficult for users to use, specifically with respect to accessing hard-to-reach areas around the eyes. Moreover, most users are a bit apprehensive about any type of treatment near the eyes.

In particular, users are typically concerned about even the slightest bit of invasiveness around the eyes. In other words, safety is paramount. Yet, some apparatuses configured for home use do not provide enough durability to prevent breakage, thereby leading to safety concerns.

### SUMMARY

In one embodiment, a derma roller apparatus includes a handle, a plurality of inner stabilizers, and a head connected to the handle via the plurality of inner stabilizers. The head includes an axial member positioned transverse to the handle. Furthermore, the head includes a left geometrically-shaped member positioned around the axial member to the left of the plurality of inner stabilizers. Additionally, the head includes a first plurality of needles situated around the left geometrically-shaped member. Moreover, the head includes a right geometrically-shaped member positioned around the axial member to the right of the plurality of inner stabilizers. Also, the head includes a second plurality of needles situated around the right geometrically-shaped member.

In another embodiment, the derma roller apparatus has a left axial member and a right axial member. Each of the left axial member and the right axial member is positioned transverse to the handle.

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1 illustrates a multi-pronged inner stabilizer configuration for a derma roller apparatus.

FIG. 2 illustrates an interspersed geometrically-shaped member that is positioned in between the left geometrically-shaped member, the right geometrically-shaped member, and at least two of the plurality of inner stabilizers.

FIG. 3A illustrates an assembled version of the derma roller apparatus.

FIG. 3B illustrates a left end cap and a right end cap that are utilized to maintain the geometrically-shaped members on an axial member.

FIG. 4 illustrates a derma roller apparatus that may have five inner stabilizers.

FIG. 5 illustrates a derma roller apparatus with a head that has no curvature.

FIG. 6 illustrates a derma roller apparatus with a head that has no curvature having as many as five inner stabilizers.

FIG. 7 illustrates a user utilizing the derma roller apparatus illustrated in FIGS. 3A and 3B.

FIG. 8A illustrates a swivel that is utilized to move the head with respect to the handle along a yaw axis in a rightward motion.

FIG. 8B illustrates the swivel of FIG. 8A in a neutral position.

FIG. 8C illustrates the swivel that is utilized to move the head with respect to the handle along a yaw axis in a leftward motion.

FIG. 9A illustrates a swivel that is utilized to move the head with respect to the handle along a pitch axis in an upward motion.

FIG. 9B illustrates the swivel of FIG. 9A in a neutral position.

FIG. 9C illustrates the swivel that is utilized to move the head with respect to the handle along a pitch axis in a downward motion.

FIG. 10A illustrates a swivel that is utilized to move the head with respect to the handle along a roll axis in a rightward motion.

FIG. 10B illustrates the swivel of FIG. 10A in a neutral position.

FIG. 10C illustrates the swivel that is utilized to move the head with respect to the handle along a roll axis in a leftward motion.

FIG. 11 illustrates the user utilizing the derma roller apparatus illustrated in FIGS. 8A-8C.

FIG. 12A illustrates a swivel that may be a ball and socket mechanism that is configured to move the handle along one or more axes with respect to the head.

FIG. 12B illustrates a neutral position of the head.

FIG. 12C illustrates a roll movement of the head.

FIG. 12D illustrates a downward pitch movement and a roll movement of the head.

FIG. 12E illustrates a downward pitch movement and a roll movement of the head.

### DETAILED DESCRIPTION

A multi-pronged inner stabilizer configuration is provided for a derma roller apparatus. The multi-pronged inner stabilizer configuration may be utilized for a variety of derma rollers, particularly with respect to eye derma rollers and face derma rollers. The term inner stabilizer connotes a member that connects a handle to a portion of an axial member, whereby the portion is within the wingspan of the head of the derma roller. A stabilizer stabilizes a head during motion of a handle of the derma roller such that the needles positioned around the head are substantially confined to rotational movement around the head during application of the derma roller to the skin, rather than other movements such as transverse movements in unintended directions.

In contrast with previous configurations, the multi-pronged inner stabilizer configuration provides access to hard-to-reach areas around the eyes and on the face, while also maximizing durability during usage. The inner stabilizers stabilize the head of the derma roller with respect to the handle, but also allow for access by needles situated around the derma roller head to hard-to-reach areas, which was previously difficult to obtain via stabilizers positioned outside the wingspan, or at the outer extremities, of the derma roller head. In essence, previous configurations had outer stabilizers that blocked the derma roller needles from puncturing hard-to-reach areas of the skin (e.g., by the corners of the eyes, edges of the nose, etc.). As a result, the derma roller apparatus allows for improved collagen production in the eye area of a user, thereby providing the potential for improved skincare rejuvenation to help reduce or eliminate eye skin conditions such as crow's feet, dark circles, laugh lines, etc.

By having multiple prongs, rather than one prong, the multi-pronged inner stabilizer configuration minimizes the possibility of flexing or breakage during rolling of the needles around the head with respect to the skin of a user. As a result, the multi-pronged inner stabilizer configuration enhances the safety of derma rolling in sensitive areas, such as around the eyes and the nose.

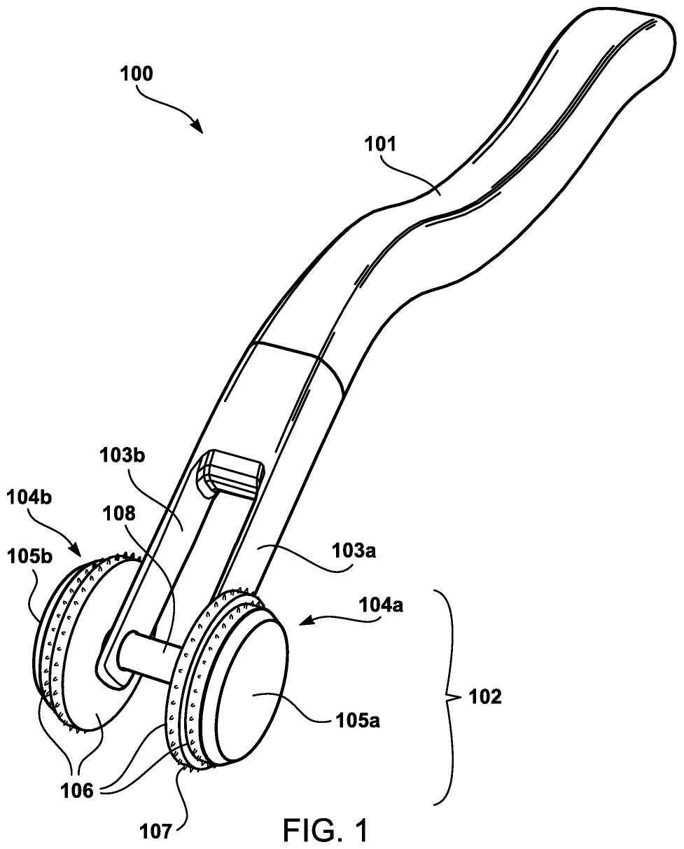

FIG. 1 illustrates a multi-pronged inner stabilizer configuration for a derma roller apparatus 100. As opposed to having obstructive outer stabilizers, the derma roller apparatus 100 has a plurality of inner stabilizers 103a and 103b that connect a handle 101 to a head 102 via an axial member 109, which is positioned transverse to the handle 101. Furthermore, a left geometrically-shaped member 104b is positioned around the axial member 109 to the left of the plurality of inner stabilizers 103a and 103b, and a right geometrically-shaped member 104a is positioned around the axial member 109 to the right of the plurality of inners stabilizers 103a and 103b. Furthermore, a plurality of needles 107 is positioned around each of the right geometrically-shaped member 104a and the left geometrically-shaped member 104b. (The phrase geometrically-shaped member is intended to include individual members (e.g., a single disc-shaped member 106) or a group of members (e.g., multiple discs 106 that form a geometric shape, such as an ellipsoid or a sphere).) The geometric shape formed by one or more discs may be substantially (e.g., perceived by a human as being in the shape of an ellipsoid or sphere).

The derma roller apparatus 100 may be optimized for convenient access to the skin around the eyes of the user or wrinkles by the nose of a user. In other words, the derma roller apparatus 100 may be constructed according to varying widths most suitable for a particular function. For example, the derma roller apparatus 100 may be constructed to have a width of the head in the range of one quarter inch and one inch, thereby being an eye derma roller apparatus. As another example, the derma roller apparatus 100 may be constructed to have a width of the head in the range of one half inch and two inches, thereby being a face derma roller. (The ranges provided for herein are examples—slight variations (e.g., within zero to twenty percent) may be utilized instead.) As another example, the inner stabilizers 103a and 103b each may have a width of approximately one half millimeter to five millimeters. (The width of the inner stabilizer 103a and 103b are provided only as examples; other widths may be utilized instead.)

Furthermore, in one embodiment, as illustrated in FIG. 1, the left geometrically-shaped member 104b has a diameter that progressively diminishes from a first end of the left geometrically-shaped member 104b (i.e., the end toward an end cap 105b) to a second end of the left geometrically-shaped member 104b (i.e., the end toward the plurality of inner stabilizers 103a and 103b). Similarly, the right geometrically-shaped member 104a has a diameter that progressively diminishes from a first end of the right geometrically-shaped member 104a (i.e., the end toward an end cap 105a) to a second end of the right geometrically-shaped member 104a (i.e., the end toward the plurality of inner stabilizers 103a and 103b).

A plurality of needles 107 are situated around the geometrically-shaped members 104a and 104b. The plurality of needles 107 may have a length that is in a range of twenty-five tenths millimeters to two and one half millimeters.

In the illustrated embodiment, the handle 101 is fixed with respect to the head 102. However, in alternative embodiments, the handle 101 may be movable (e.g., via a swivel) with respect to the head 102.

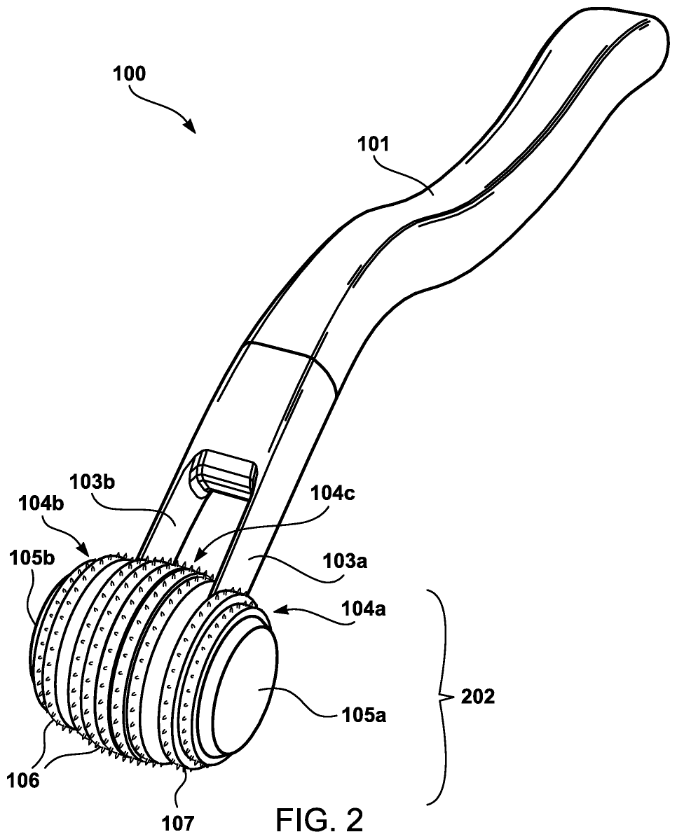

Furthermore, in the illustrated embodiment, the portion of the axial member 109 that is positioned in between the right inner stabilizer 103a and the left inner stabilizer 103b is not surrounded by a geometrically-shaped member. By way of contrast, FIG. 2 illustrates an interspersed geometrically-shaped member 104c that is positioned in between the left geometrically-shaped member 104b, the right geometrically-shaped member 104a, and at least two of the plurality of inner stabilizers 103a and 103b. The interspersed geometrically-shaped member 104c has one or more additional pluralities of needles 107 positioned around thereon. (Although one interspersed geometrically-shaped member 104c is illustrated, additional interspersed geometrically-shaped members may be utilized instead.)

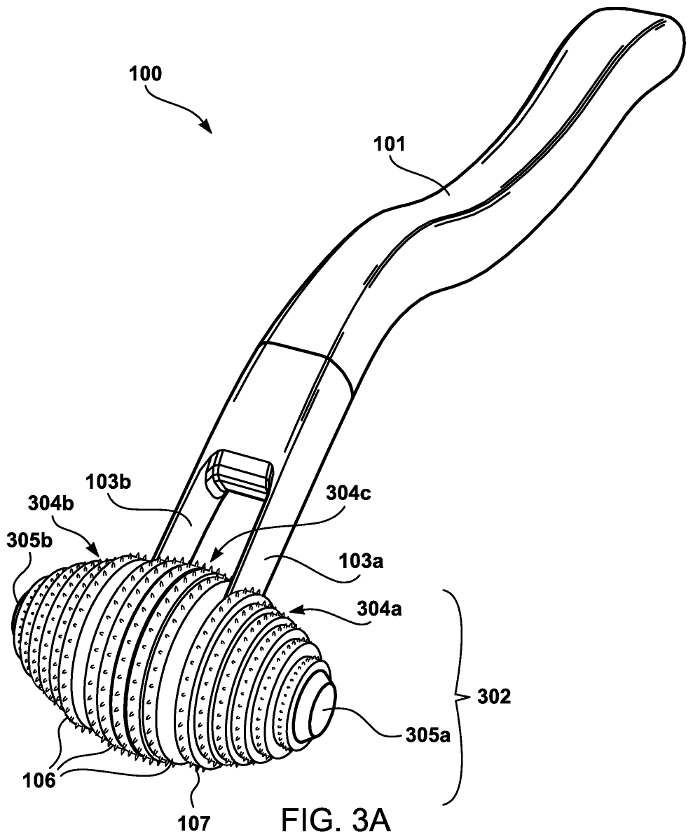

FIGS. 3A and 3B illustrate the derma roller apparatus 100 of FIGS. 1 and 2, but with a head 302 that has more overall curvature (e.g., ellipsoid shape) than the head 102 with a substantially cylindrical shape. The increased curvature may allow for enhanced ease-of-access to hard-to-reach areas. In particular, FIG. 3A illustrates an assembled version of the derma roller apparatus 100. A left end cap 305b and a right end cap 305a are utilized to maintain the geometrically-shaped members 304a-c on an axial member 305a, as illustrated in FIG. 3B. Various bearings 310 may be utilized to allow insertion of the axial member 309 through the geometrically-shaped members 304a-c.

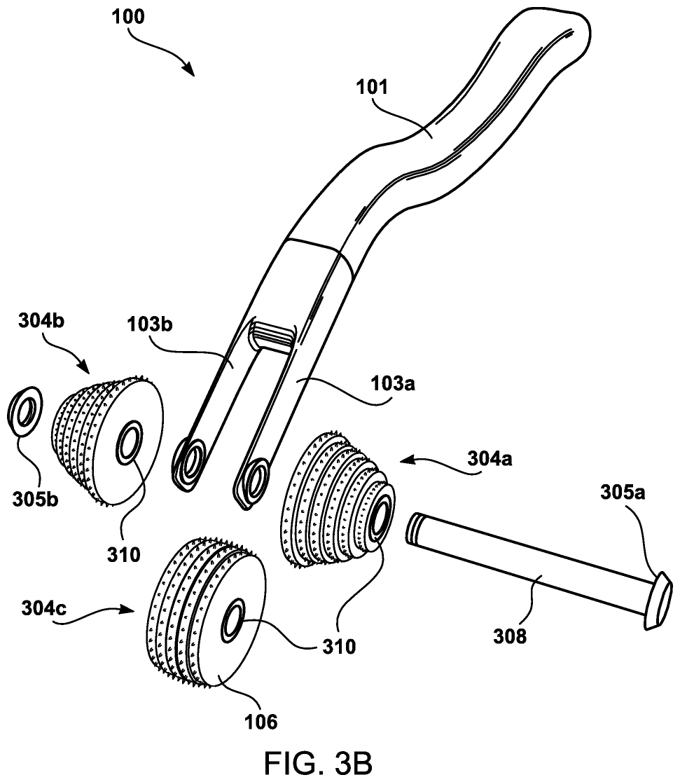

Although the quantity of inner stabilizers in FIGS. 1-3B is illustrated as two, the derma roller apparatus 100 may have a quantity of inner stabilizers in the range of two to five. Accordingly, as an example, FIG. 4 illustrates a derma roller apparatus 400 that may have five inner stabilizers 103a-103e. Furthermore, the derma roller apparatus 400 may have a head 402 that has one or more interspersed geometrically-shaped members.

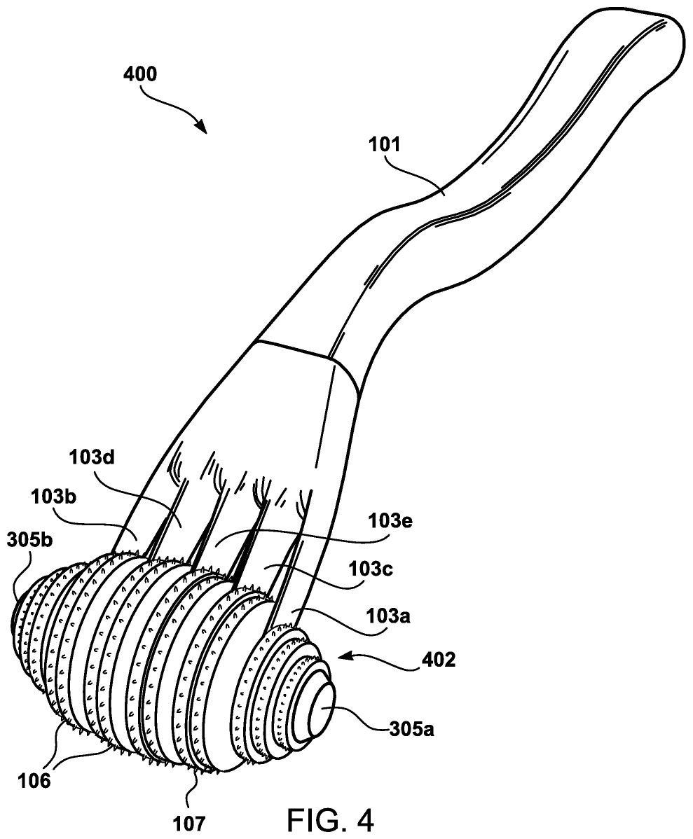

The head of the derma roller apparatus 100 is not limited to having a curvature. FIG. 5 illustrates a derma roller apparatus 500 with a head 502 that has no curvature. In other words, the geometrically-shaped members all have an equal diameter (e.g., cylindrically-shaped discs with an equal diameter). As an example, two inner stabilizers 103a and 103b are illustrated. However, as illustrated, a derma roller apparatus 600 with a head 602 that has no curvature may have as many as five inner stabilizers 203a-203e, as illustrated in FIG. 6.

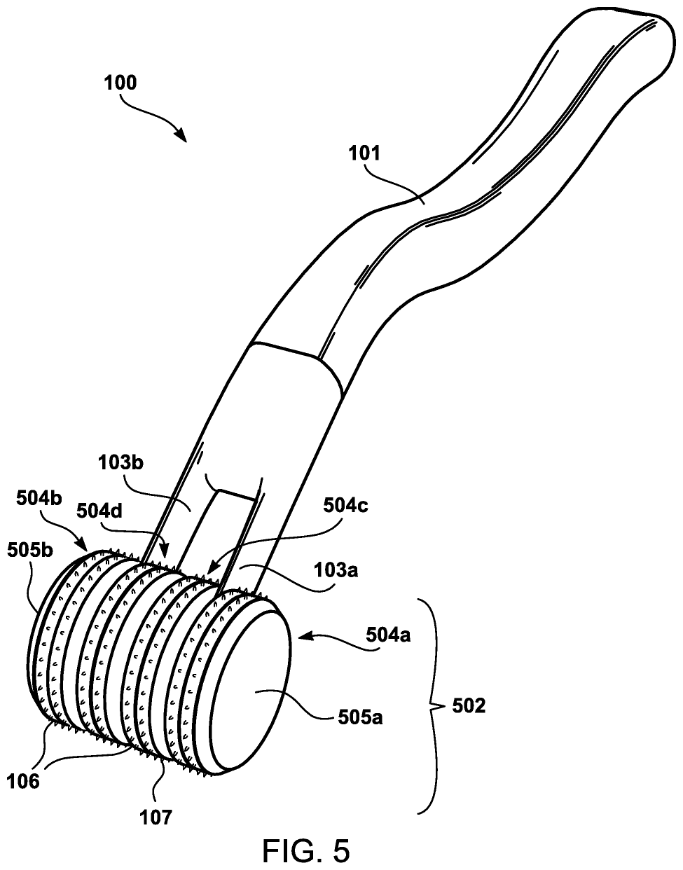

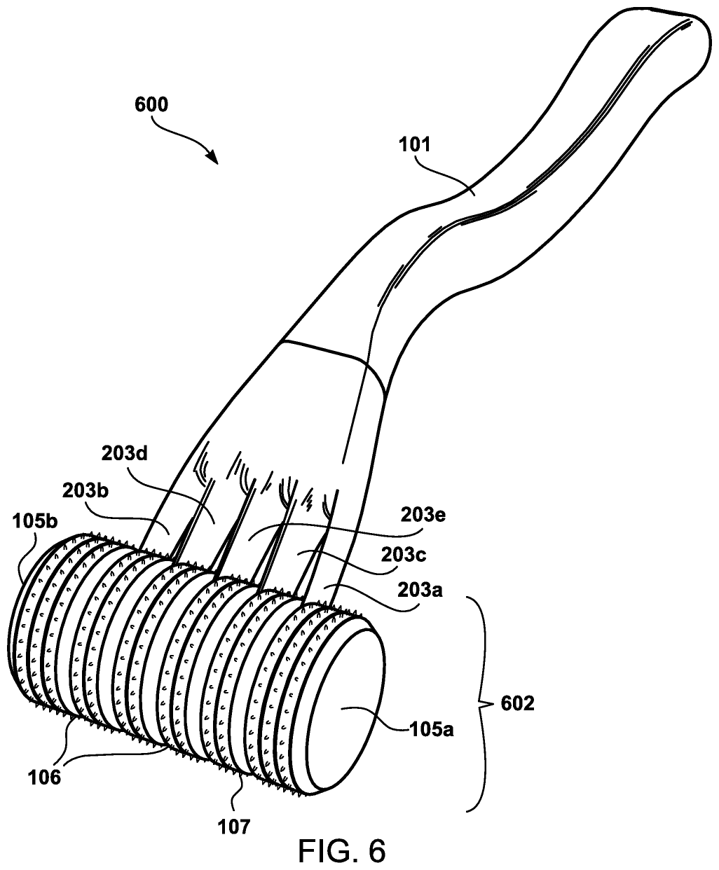

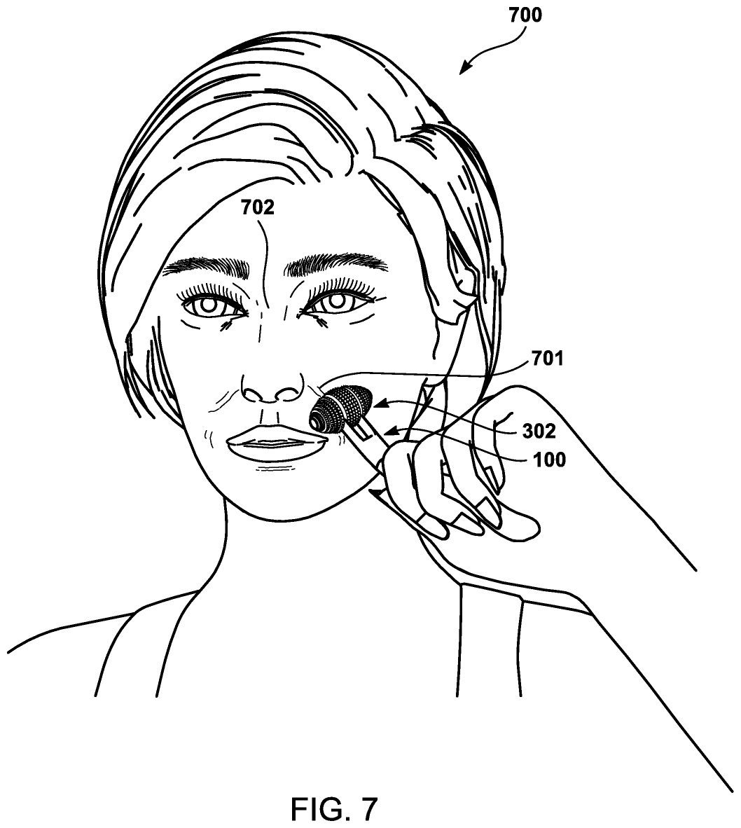

FIG. 7 illustrates a user 700 utilizing the derma roller apparatus 100 illustrated in FIGS. 3A and 3B. In particular, the user 700 is able to utilize the derma roller apparatus 100 as a face derma roller. For example the user 700 may move the derma roller apparatus with respect to one or more laugh lines 701 to attempt to remove laugh lines and wrinkles in the face area. Given the difficulty accessing the edges around the nose 702, an area in close proximity to the laugh line 701, the use of the inner stabilizers 103a and 103b helps allow for access to the edges around the nose 702 that was previously prevented by having an outer stabilizer in that area.

Although the particular example illustrated above utilizes a particular quantity of inner stabilizers and had a particular head curvature, any of the other configurations provided for herein may be utilized instead. Furthermore, a different sized derma roller apparatus 100 may be utilized as an eye derma roller for access to the corners of the eyes, eye lids, crow's feet, etc.

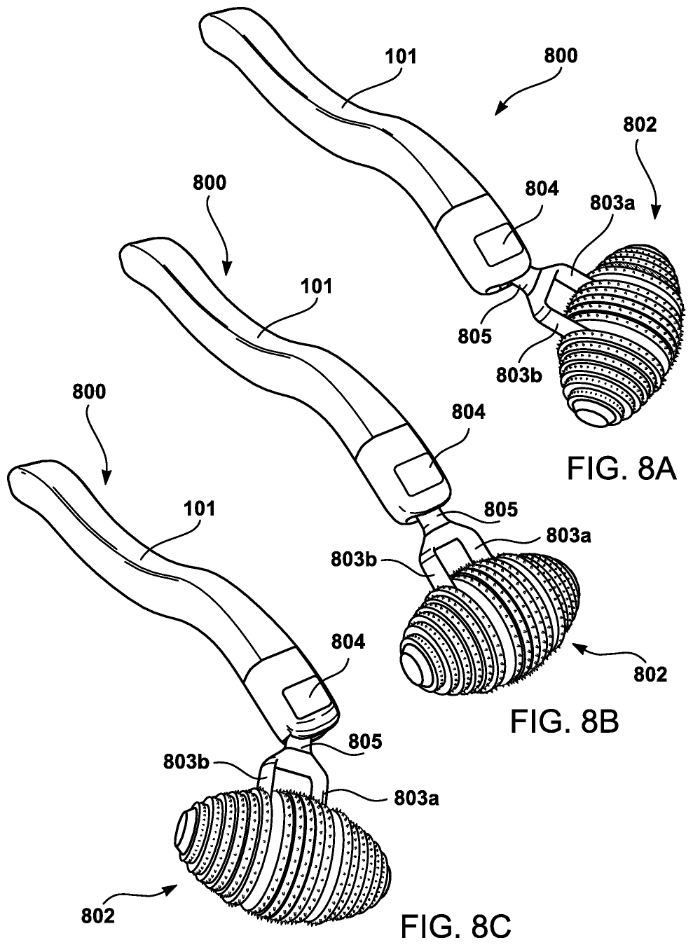

Although the previous embodiments were illustrated as having the handle 101 fixed with respect to the head 102 and the inner stabilizers 103a and 103b, in an alternative embodiment illustrated in FIGS. 8A-8B, a swivel 805 may be configured to effectuate movement of the handle 101 with respect to the head 802. In particular, FIG. 8A illustrates a swivel 805 that is utilized to move the head 802 with respect to the handle 101 along a yaw axis in a rightward motion. In one embodiment, the inner stabilizers 803a and 803b are connected to the head 802 and also the swivel 805. Further, in one embodiment, an actuator 804 (e.g., button) may be utilized to lock the head 802 into a particular position. For example, the actuator 804 may be positioned on the handle 101, and may allow the head 802 to be locked into a particular angular position with respect to the handle 101, thereby allowing the user 700 to have the convenience of adjusting the head 802 to a particular angle for enhanced access to hard-to-reach areas. FIG. 8B illustrates the swivel 805 of FIG. 8B in a neutral position. Finally, FIG. 8C illustrates the swivel 805 that is utilized to move the head 802 with respect to the handle 101 along a yaw axis in a leftward motion.

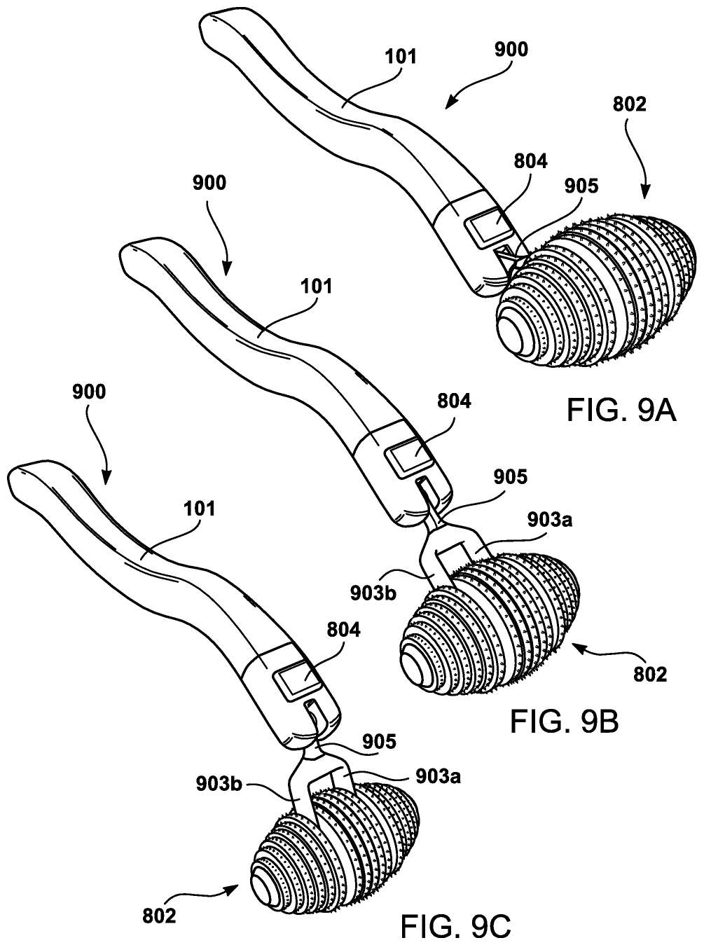

FIGS. 9A-9C illustrate a swivel 905 that is configured to effectuate movement of the handle 101 with respect to the head 802 along a pitch axis. In particular, FIG. 9A illustrates a swivel 905 that is utilized to move the head 802 with respect to the handle 101 along a pitch axis in an upward motion via the inner stabilizers 903a and 903b. FIG. 9B illustrates the swivel 905 of FIG. 9A in a neutral position. Finally, FIG. 9C illustrates the swivel 905 that is utilized to move the head 802 with respect to the handle 101 along a pitch axis in a downward motion.

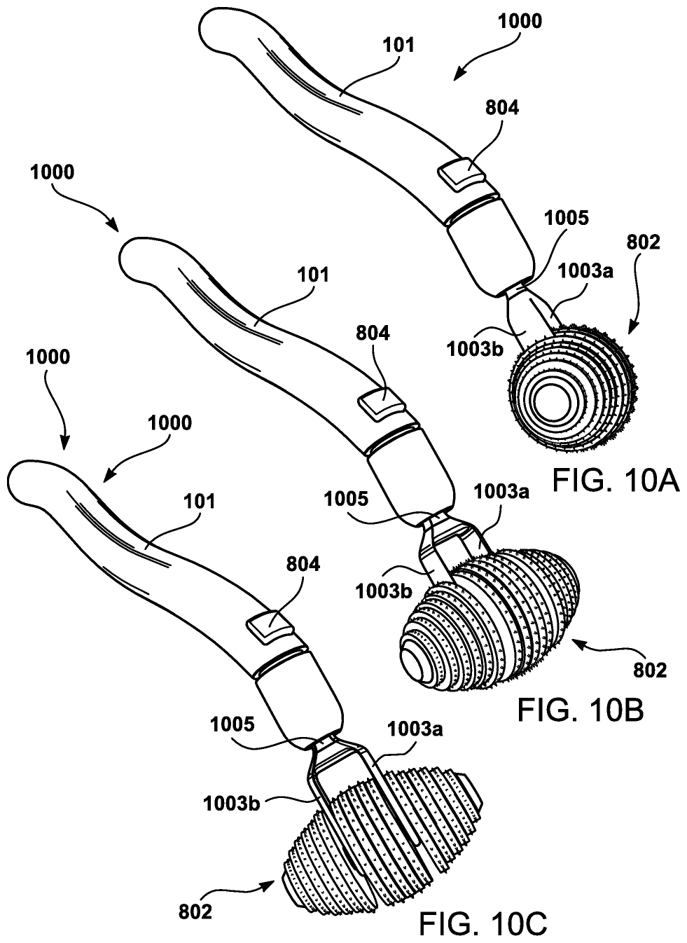

Moreover, FIGS. 10A-10C illustrate a swivel 1005 that is configured to effectuate movement of the handle 101 with respect to the head 802 along a roll axis via the inner stabilizers 1003a and 1003b. In particular, FIG. 10A illustrates a swivel 1005 that is utilized to move the head 802 with respect to the handle 101 along a roll axis in a rightward motion. FIG. 10B illustrates the swivel 1003 of FIG. 10A in a neutral position. Finally, FIG. 10C illustrates the swivel 905 that is utilized to move the head 802 with respect to the handle 101 along a roll axis in a leftward motion.

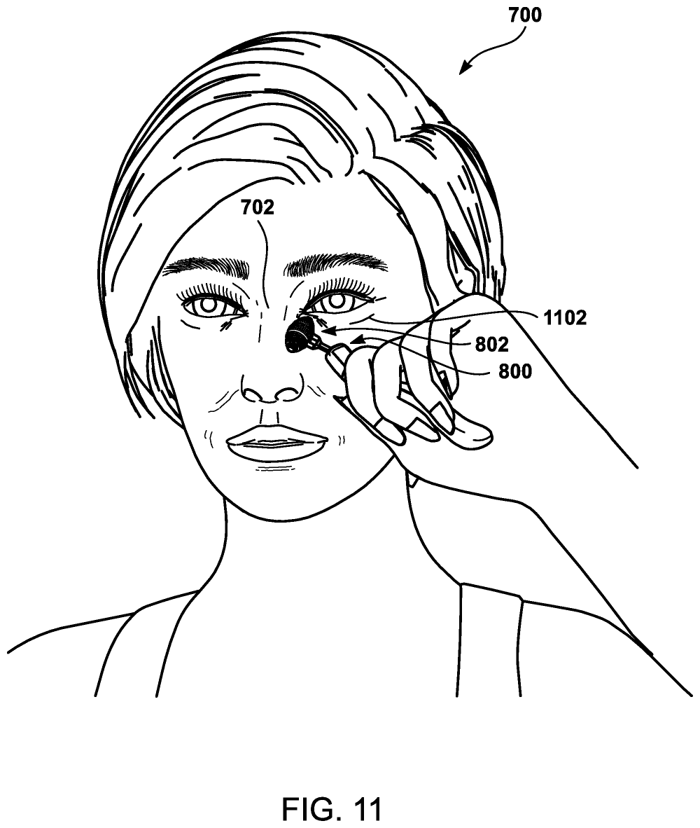

FIG. 11 illustrates the user 700 utilizing the derma roller apparatus 800 illustrated in FIGS. 8A-8C. In particular, the user 700 is able to utilize the derma roller apparatus 800 as an eye derma roller. For example the user 700 may move the derma roller apparatus 800 with respect to crows' feet 1102 by an edge of the nose 702. The user 700 is able to adjust the angle of the head 802 to access the hard-to-reach edge by the nose 702, which could be quite difficult if an outer stabilizer was present. (Other types of derma rollers with access to different axes, such as those illustrated in FIGS. 9A-10C, may be utilized instead.)

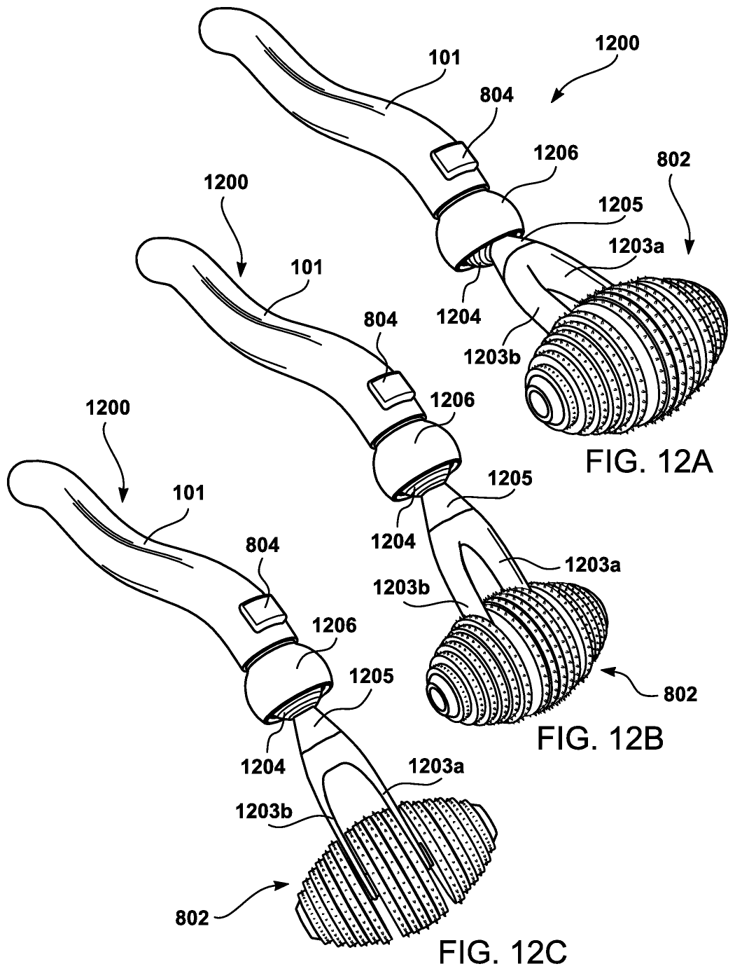

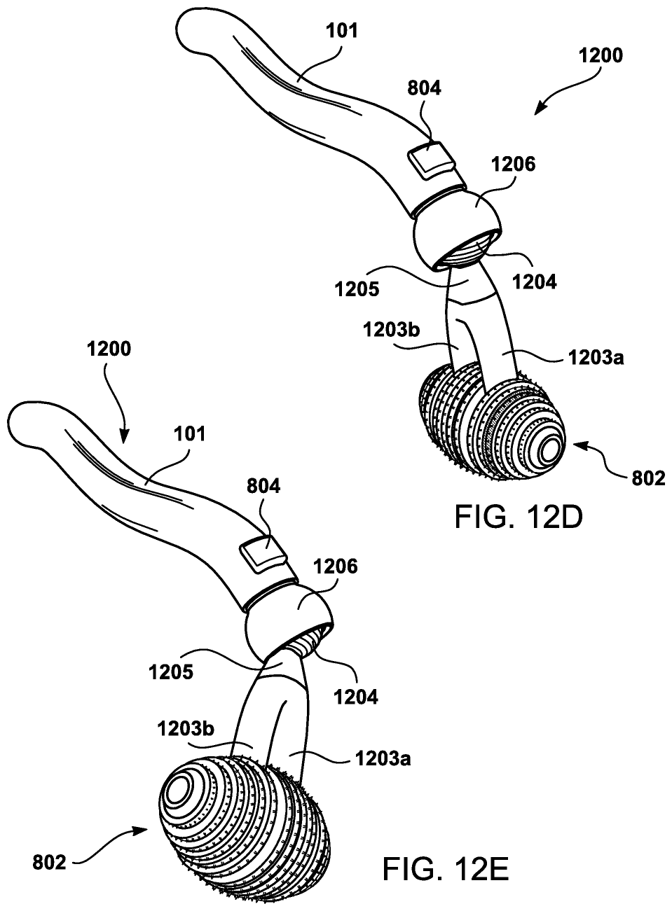

FIGS. 12A-12E illustrate a derma roller apparatus 1200 with free range (e.g., three hundred sixty degrees movement). As an example, as illustrated in FIG. 12A, a swivel 1205 may be a ball and socket mechanism that is configured to move the handle along one or more axes with respect to the head 802 via inner stabilizers 1203a and 1203b. For example, the swivel 1204 may allow for an upward pitch movement. For instance, a ball 1204 may be inserted into a socket within an outer housing 1206 connected to the handle 101. As a result, the swivel 1205 allows for free range movement (e.g., three hundred sixty degrees movement). In essence, the head 802 may be adjusted along a single axis (e.g., yaw, pitch, or roll), or along multiple axes (e.g., yaw, pitch, and roll). FIG. 12B illustrates a neutral position of the head 802. Furthermore, FIG. 12C illustrates a roll movement of the head 802. FIG. 12D illustrates a downward pitch movement and a roll movement of the head 802. Finally, FIG. 12E illustrates a downward pitch movement and a roll movement of the head 802.

It is understood that the apparatuses described herein may also be applied in other types of apparatuses. Those skilled in the art will appreciate that the various adaptations and modifications of the embodiments of the apparatuses described herein may be configured without departing from the scope and spirit of the present processes and systems. Therefore, it is to be understood that, within the scope of the appended claims, the present apparatuses may be practiced other than as specifically described herein.

I claim:

1. A derma roller apparatus comprising:
a handle;
a plurality of inner stabilizers; and
a head connected to the handle via the plurality of inner stabilizers, the head comprising:
an axial member positioned transverse to the handle,
a left geometrically-shaped member positioned around the axial member to the left of the plurality of inner stabilizers,
a first plurality of needles situated around the left geometrically-shaped member,
a right geometrically-shaped member positioned around the axial member to the right of the plurality of inner stabilizers,
a second plurality of needles situated around the right geometrically-shaped member.

2. The derma roller apparatus of claim 1, wherein the left geometrically-shaped member has a diameter that progressively diminishes from a first end of the left geometrically-shaped member to a second end of the left geometrically-shaped member, the first end of the left geometrically-shaped member being proximal to the plurality of inner stabilizers, the second end of the left geometrically-shaped member being distal to the plurality of inner stabilizers.

3. The derma roller apparatus of claim 1, wherein the right geometrically-shaped member has a diameter that progressively diminishes from a first end of the right geometrically-shaped member to a second end of the right geometrically-shaped member, the first end of the right geometrically-shaped member being proximal to the plurality of inner stabilizers, the second end of the right geometrically-shaped member being distal to the plurality of inner stabilizers.

4. The derma roller apparatus of claim 1, wherein the left geometrically-shaped member has a diameter that is equal to a diameter of the right geometrically-shaped member.

5. The derma roller apparatus of claim 1, wherein each of the plurality of inner stabilizers connects to a corresponding portion of the axial member that is within a width of the axial member.

6. The derma roller apparatus of claim 1, wherein the plurality of inner stabilizers has a quantity in a range of two to five inner stabilizers.

7. The derma roller apparatus of claim 1, wherein the head further comprises one or more interspersed geometrically-shaped members positioned in between the left geometrically-shaped member, the right geometrically-shaped member, and at least two of the plurality of inner stabilizers, the one or more interspersed geometrically-shaped members having one or more additional pluralities of needles positioned around thereon.

8. The derma roller apparatus of claim 7, wherein each of the first plurality of needles has a length in a range of twenty-five tenths millimeters to two and one half millimeters, each of the second plurality of needles has a length in a range of twenty-five tenths millimeters to two and one half millimeters, and each of the additional pluralities of needles has a length in a range of twenty-five tenths millimeters to two and one half millimeters.

9. The derma roller apparatus of claim 1, wherein the handle is fixed with respect to the head via the plurality of inner stabilizers.

10. The derma roller apparatus of claim 1, further comprising a swivel that is configured to effectuate movement of the handle with respect to the head.

11. The derma roller apparatus of claim 10, further comprising a locking mechanism that restricts the swivel from movement at an angle between the head and the handle.

12. The derma roller apparatus of claim 11, wherein the handle comprises an actuator that activates the locking mechanism.

13. The derma roller apparatus of claim 10, wherein the swivel is connected between the handle and the plurality of inner stabilizers.

14. The derma roller apparatus of claim 10, wherein the swivel is a ball and socket mechanism that is configured to move the handle along multiple axes with respect to the head.

15. The derma roller apparatus of claim 10, wherein the swivel is configured to rotate the handle only about a yaw axis with respect to the head without rotation about a pitch axis and a roll axis.

16. The derma roller apparatus of claim 10, wherein the swivel is configured to rotate the handle only about a pitch axis with respect to the head without rotation about a yaw axis and a roll axis.

17. The derma roller apparatus of claim 10, wherein the swivel is configured to rotate the handle only about a roll axis with respect to the head without rotation about a pitch axis and a yaw axis.

18. The derma roller apparatus of claim 10, wherein a width of the head is in an eye derma roller specific range between one quarter inch and one inch.

19. The derma roller apparatus of claim 10, wherein a width of the head is in a face derma roller specific range between one half inch and two inches.

20. A derma roller apparatus comprising:
a handle;
a plurality of inner stabilizers; and
a head connected to the handle via the plurality of inner stabilizers, the head comprising:
a left axial member positioned transverse to the handle,
a left geometrically-shaped member positioned around the left axial member to the left of the plurality of inner stabilizers,
a first plurality of needles situated around the left geometrically-shaped member,
a right axial member positioned transverse to the handle,
a right geometrically-shaped member positioned around the axial member to the right of the plurality of inner stabilizers,
a second plurality of needles situated around the right geometrically-shaped member.
